(19) United States
(12) Patent Application Publication
(10) Pub. No.: US 2015/0174009 A1
(43) Pub. Date: Jun. 25, 2015

(54) MULTI-LAYER CAPSULE AND MANUFACTURE METHOD THEREOF

(71) Applicants: Ikuo Goto, Nara (JP); Chin-Chih Chiang, West Covina, CA (US); Yen-Fei Chen, Taichung City (TW)

(72) Inventors: Ikuo Goto, Nara (JP); Chin-Chih Chiang, West Covina, CA (US); Yen-Fei Chen, Taichung City (TW)

(73) Assignee: Orient Pharma Co., Ltd., Yunlin County 63247 (TW)

(21) Appl. No.: 13/512,012

(22) PCT Filed: Jun. 15, 2011
(86) PCT No.: PCT/US11/40589

§ 371 Date: Aug. 31, 2012

(51) Int. Cl. A61J 3/07; B05D 1/00; A61K 38/14; A61K 9/48; A61K 31/519

(52) U.S. Cl. CPC A61J 3/071; A61K 9/4808; A61K 31/519; A61K 38/14; A61K 9/4833; B05D 1/00; B05D 2259/00

(57) ABSTRACT

The present invention provides multi-layer capsules and manufacture methods thereof Different materials are added in sequence into a capsule to form a multi-layer capsule without diffusion and/or interaction between layers.

### BACKGROUND OF THE INVENTION

In the manufacture of pharmaceuticals, encapsulation refers to a range of techniques used to enclose medicines in a relatively stable shell known as a capsule. The two main types of capsules are hard-shelled capsules and soft-shelled capsules. For example, FIG. 1A show a common hard-shelled capsule formed by a tubular body to hold ingredients and a tubular cover body (cap). The tubular body is smaller than the tubular cover body as shown; however, the body can be larger than the cover body. The capsules are generally supplied in a prelocked condition, i.e., a condition in which the capsule cover body is mounted on the capsule body, but can readily be removed therefrom at any desired or required time for, for example, adding of the filler material thereinto. In the processes of adding ingredients, the prelocked capsules are loaded into a hopper; a predefined number of capsules are then released followed by separation of the cover body from the body of capsules to fill ingredients. After the adding, the cover body is put back onto the body of the capsule. Depending on the desired purposes under different circumstances, such as deodorization, leak prevention, anti-oxidation, the capsules may undergo various processes of drying steps.

There are several capsule adding devices on the market that can handle granules, or powder adding without many problems. However, these devices have problems maintaining desired drug efficacy when dealing with semi-solid or liquid ingredients. For example, it is important to maintain a steady level of separating surface of the first filler in a form of semi-solid or liquid so the first filler will not mix with the next ingredients of semi-solid or liquid. Once mixed, the drug efficacy will suffer due to diffusion of drugs or drug-drug interactions.

In addition, if the separating surface of the semi-solid or liquid is foaming or not fixed, the drug on or close to the separating surface will dissolve differently from the rest; this is due to the larger contacting surface between layers from the uneven, tilted or foaming surface. The drug dissolution time become unpredictable and the expected efficacy cannot be achieved.

### SUMMARY OF THE INVENTION

The present invention provides multi-layer capsules and methods of manufacturing same. The invention multi-layer capsules comprise at least two layers of homogeneous materials and at least one barrier layer where each layer of homogenous material comprises the same or different biopharmaceutical ingredient wherein each layer of homogeneous material has the same or different form of liquation from other layers. The multi-layer capsules, in some embodiments, comprise:
a body comprising at least one opening and an internal space;
at least one cover body to mount or dismount over the at least one opening of the body;
a barrier layer disposed in the internal space of the body to separate the first compartment and the second compartment wherein the barrier layer is solid at room temperature and semi-solid or liquid at a temperature higher than 35° C.

In some embodiments, provided herein are methods of manufacturing a multi-layer capsule, the methods comprise
providing a capsule comprising a body, and at least one cover body wherein the body has an internal space and at least one opening;
adding a first homogeneous material, a second homogeneous material, and a barrier layer to the internal space of the body wherein the barrier layer is added before or after the first homogeneous material; and
mounting the at least one cover body over the at least one opening of the body, wherein the first and second homogeneous material is separated by the barrier layer.

The methods of manufacturing the invention multi-layer capsules, in some embodiments, comprise adding a barrier layer in a semi-sold or liquid form to a body of a capsule to separate a first compartment and a second compartment wherein the barrier layer is solid at a temperature lower than 35° C. The barrier layer, in some embodiments, comprises mineral oil and paraffin wax in a weight ratio between 0 to 4.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention arc set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1C show illustrative schematic drawing of prior art capsules and the invention capsules.

FIG. 2 shows exemplary flow chart for the preparation of invention capsules.

FIG. 3 shows an illustrative diagram for manufacturing the invention multi-layer capsules.

FIG. 4 shows a simplified illustrative diagram for preparation of a single invention multi-layer capsule.

FIG. 5A and 5B shows an illustrative invention nozzle with specific spreading angles.

FIG. 6A-6B show an illustrative diagram from another embodiment for manufacturing invention multi-layer capsules.

FIG. 7A-7B are exemplary flow chart based on FIG. 6A/6B manufacturing diagram.

### DETAILED DESCRIPTION OF THE INVENTION

In some embodiments, the present invention provides multi-layer capsules comprising at least two layers of homogeneous materials and at least one barrier layer where each layer of homogenous material comprises the same or different biopharmaceutical ingredient (e.g. an active pharmaceutical ingredient (API), a dietary supplements ingredient, and the like) wherein each layer of homogeneous material has the same or different form of liquation from other layers. In order to achieve drug stability in the capsule and the desired drug efficacy as well as to make sure the drug(s) do not interact during preparation of the multi-layer capsules, the invention capsules further comprise a barrier component consisting of stable and bio-friendly ingredients.

The active pharmaceutical ingredients may be antibiotics such as vancomycin, teicoplanin, ramoplanin, difimicin, kanamycin, neomycin, colistin, and the like, hypnotic drugs such as zaleplon, zolpidem, and the like, or other non-limited pharmaceutical ingredients. The dietary supplements ingredients may be vitamins, amino acids, botanical extracts, nonbotanicals, or other non-limited dietary supplements.

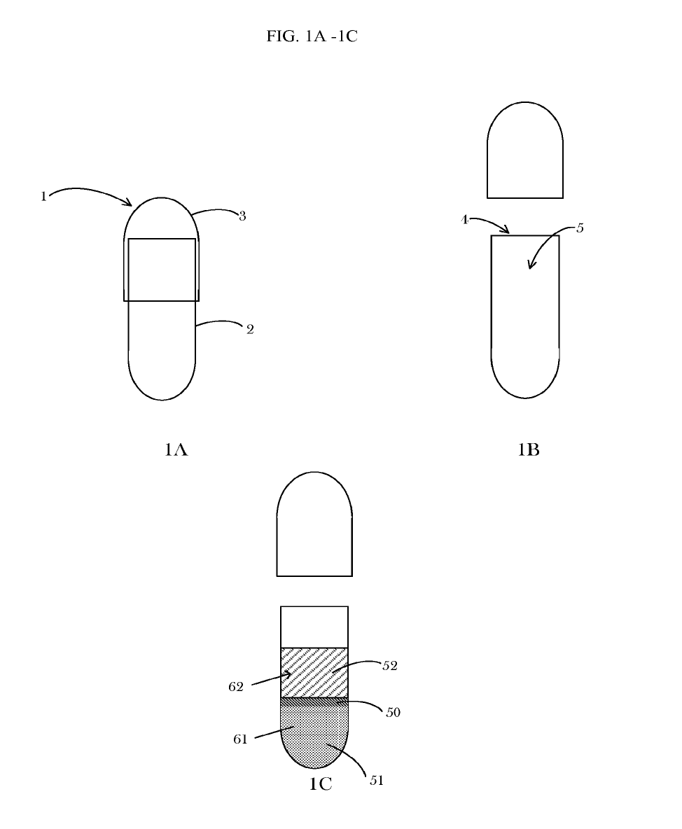

Referring to FIGS. 1A-1C, the invention capsule 1 comprises at least one cover body 3 and a body 2 where the body 2 has at least one opening 4 and an internal space 5. In some embodiments, the cover body 3 has a slightly larger diameter than the body 2 allowing the cover body 3 to mount over the opening of the body 2 and close the opening 4. In some embodiments, the body 2 comprises a barrier layer 50 that connects with an inner wall of the body 2 separating the internal space 5 to provide the first compartment 61 and the second compartment 62 (FIG. 1C). In certain embodiments, the thickness of the barrier layer accounts for about 5% to 25% of the body length. The barrier layer is made of bio-friendly materials that are dissolvable, digestible and/or dischargeable in the digestive tract, under the gastrointestinal environment. In some embodiments, the barrier layers 50 comprise mineral oil, paraffin (paraffin wax), combinations thereof, or the like. In some embodiments, the weight ratio of mineral oil and paraffin wax is between 0 to 4, which means the barrier layer may comprise 0 to 80% of mineral oil mixed with paraffin wax or at least 20% wax so the barrier layer remains solid at room temperature or a temperature of normal storage conditions. Melting point of the barrier layer is determined by the ratio of mineral oil and paraffin wax.

Referring to FIG. 1C, the first compartment 61 and the second compartment 62 contain a first homogeneous material 51 and a second homogeneous material 52, respectively, where a barrier layer 50 separates the first homogeneous material 51 and the second homogeneous material 52. In other words, the internal space 5 of the body comprises three adding components. In some embodiments, the first homogeneous material 51 and the second homogeneous material 52 comprise pharmaceutical active ingredients. The first or second homogeneous material can be a liquid, solid, or semi-solid material at room temperature or a temperature of normal storage conditions. In some embodiments, the liquid comprises a homogeneous liquid or a suspension. In some embodiments, the solid comprises solid block, micro capsules, granules, or powdery solid (e.g. powder). Semi-solid is a viscous fluid that flows relatively slow compared to liquid. Solid block refers to solids that can be in a semi-solid or liquid form at high temperature (e.g., higher than 35° C.). In some embodiments, a first or second homogeneous material comprises at least one hot melt excipient in a solid block form at room temperature or a normal storage temperature and liquid or semi-solid at high temperature (e.g., higher than 35° C.). In some embodiment, the first homogeneous material 51 has a melting point higher than the melting point of the barrier layer 50 and the barrier layer 50 has a melting point higher than the melting point of the second homogenous material 52.

The exemplary hot melt excipients include, but are not limited to, polyethylene glycols (PEGs), lipophilic compounds, propylene glycol fatty esters, an optional pH-sensitive polymers (such as sodium alginate or sodium carboxymethyl cellulose), polyethylene glycol esters, and the like.

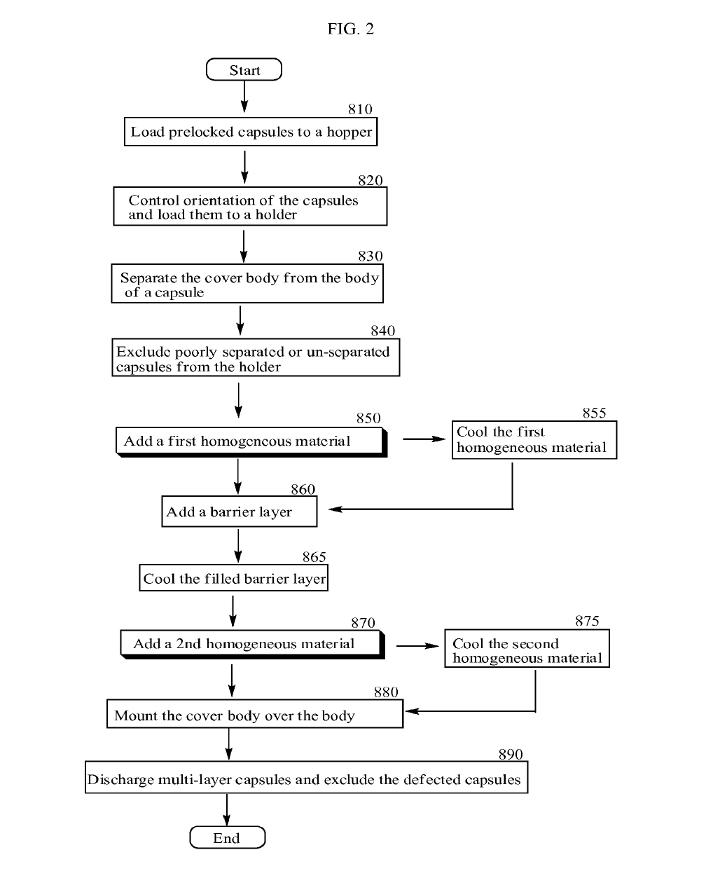

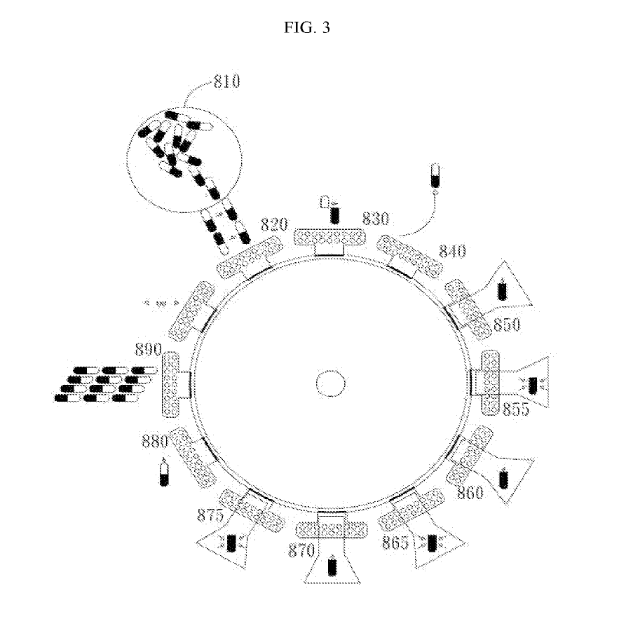

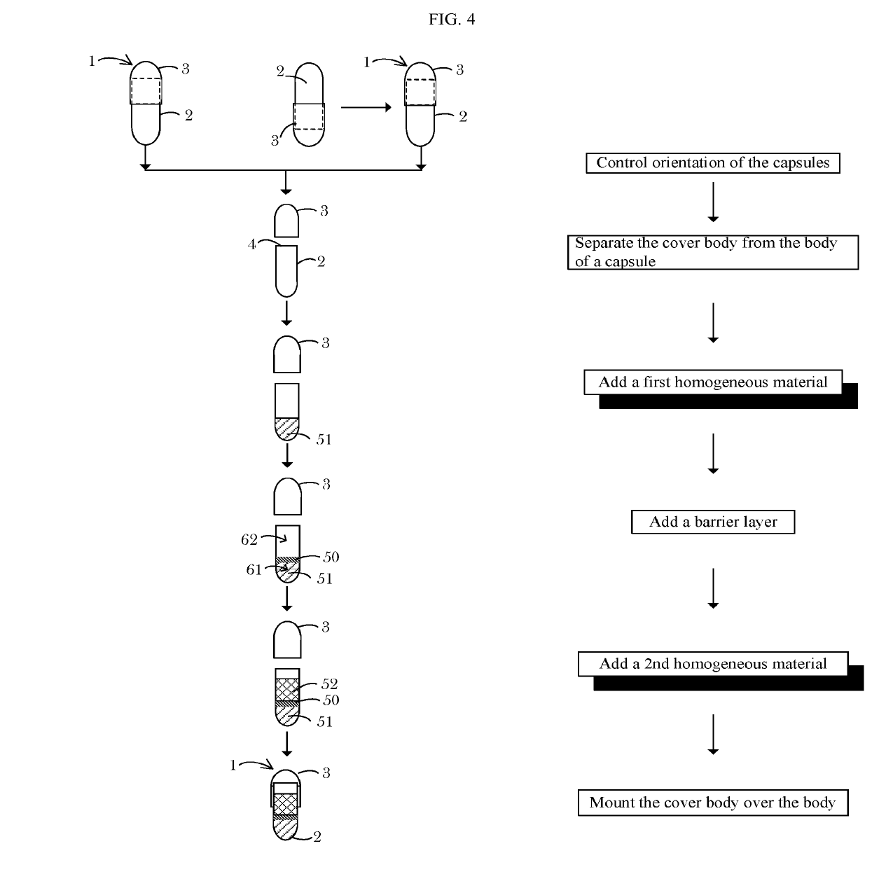

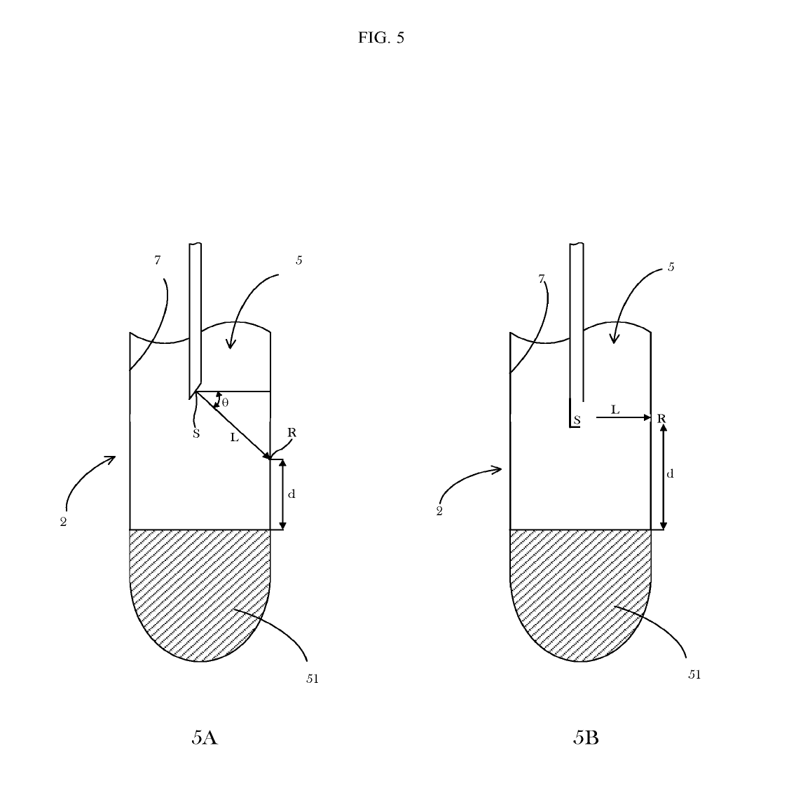

In some embodiments disclosed herein provide methods for manufacturing invention multi-layer capsules where the adding process is completed by a capsule filling device. The capsule filling device can be an intermittent and/or continuous drive to complete the capsule filling, and can be applied to the capsule holder by way of a disc link, chain link, or other suitable links known in the art. Referring to FIG. 2 and the schematic diagrams of FIGS. 3-4, step 810 includes loading prelocked capsules into a hopper. Step 820 includes loading capsules to a holder or holder block and control the orientation of the capsules. To add a first homogeneous material 51, a barrier layer 50 and a second homogeneous material 52 under fixed forms (or shapes), based on minimal contact (separating) surfaces between adjacent fillers to ensure a desired efficacy or bioavailability profile of the drugs, the orientation of the capsule for the filling process is preferred to be the same of capsule 1 as shown in FIG. 4. The axis of the body 2 and cover body 3 is perpendicular to the horizontal plane, while the opening 4 of the body is in a level and up position; this way, the interface or surface of each filler is parallel to the opening 4 under operating conditions and the adjacent fillers would have minimal contact surfaces (or separating surfaces). In some embodiments, the proper orientation of capsules can be monitored by sensors (e.g., laser or visible light sensors or the like) and adjusted accordingly. For example, the laser sensor can be used to detect forward or reverse orientation of capsules. If a capsule is inverted (i.e., the body 3 is on top), then the capsule will be rotated to forward orientation after it passes through orientation adjusted means, such as an orientation adjusting disk, or the like. If a capsule is in a position of proper orientation, the capsule will remain as is after it passes through the orientation adjusting disk. Step 830: separate the cover body 3 from the body 2 to expose the opening 4. Then, in step 840 the poor separated capsules and the defected bodies are excluded. Under a normal operation, a body 2 and a cover body 3 in a capsule will be separated accordingly. However, if the capsule does not properly separate, the un-separated capsule will be ejected by a thimble, or a thimble-like device and send to a collection box in step 840. Furthermore, in some embodiments, if a defected capsule (e.g., a capsule without the cover body or with a broken cover body or without the body or a broken body) is detected, the machine will stop operating until the defected capsule is removed by a removal means (such as manually removal of the defected capsules). The defected capsules may be detected, for example, by two laser sensors, one for detecting the body and the other one for detecting the cover body. Only when both parts are detected, the device will proceed to next step (i.e. step 850).

Step 850: add the first homogeneous material 51 into the body 2 of a capsule. The adding of the capsule may be sequential or parallel. The first homogeneous material 51, in some embodiments, comprise solid block which is semi-solid or liquid at high temperature. In certain embodiments, the solid block is semi-solid or liquid at temperature higher than 35° C. The solid block is in a solid form when the temperature is lower than 35° C. or at room temperature so it won't mix with the next adding material (e.g. the barrier layer). The first homogeneous material 51 is heated to become homogeneously semi-solid or liquid with liquation characteristics allowing it to add into the body 2 of a capsule via a adding means. In some embodiments, the adding means is via a nozzle. For example, if the melting point of the first homogeneous material 51 is A° C., and the material becomes solid block when cooled to A° C., then step 850 further includes a step to raise temperature above A° C. to make the first homogeneous material 51 in semi-solid or liquid form for easy addition via an adding means. If the first homogeneous material 51 is heated, step 855 includes cooling the first homogeneous materials, for example, to a temperature lower than A° C. via a cooling means. The cooling means includes but not limited to blowing air (room temperature or cold air) to the capsule body 2, to the capsule holder or holder block, or to the liquid or semi-solid form of the first homogeneous material 51 to speed up the cure rate of cooling. In certain embodiments, the cooling means includes applying an external cooling device; for example, comprising a refrigerant use to cool down the capsule holder so the capsule body 2 and the first homogeneous material 51 will cool down. On the other hand, if the first homogeneous material 51 can be added directly without heating such that it has the same form during the adding step and the storage step or at room temperature (e.g., powder form during the adding step and at the storage condition), step 855 needs not to proceed. The adding of the first homogeneous material 51 is completed after steps 850 and 855.

Step 860 includes adding of the barrier layer 50 into the body 2 of a capsule, to form a first compartment 61 adding with the first homogeneous material 51 and the second compartment 62 that has not yet added. In certain embodiments, the amount of bio-friendly materials used to prepare the barrier layer 51 is determined by the thickness of the barrier layer that accounts for about 5% to about 25% of the body length but not limited to this range. In some embodiments, the thickness of a barrier layer accounts for about 5% to about 20%, about 5% to about 15% or about 5% to about 10% of the body length. Furthermore, in some embodiments, the barrier layer used herein is in a semi-solid or liquid form for easy filing. As noted above, the barrier layers, in some embodiments, comprise mineral oil, paraffin (or paraffin wax), combinations thereof, and the like. In certain embodiments, the weight ratio of mineral oil and paraffin wax is between 0 to 4. When a barrier layer comprises 100% paraffin wax, the barrier layer has a melting point at about 60 to 65° C. The form of such barrier layer (100% paraffin wax), when heated to 60˜65° C., changes to a semi-solid or liquid form. The melting point of a barrier layer decreases when mineral oil is added, so the temperature required to produce a semi-solid or liquid barrier layer 51 can be lower than 60˜65° C. Therefore, the temperature used in step 860 is further determined by the melting point of the barrier layer in connection with the composition (e.g. ration of mineral oil and paraffin wax).

Furthermore, as stated before, the purpose of the barrier layer 50 is to separate the first homogeneous material 51 and the second homogeneous material 52; the barrier layer is being added after the adding of the first homogeneous material 51. Because the barrier layer is in a semi-solid or liquid form (after heated) as being added on top of the first homogeneous material 51 in a solid block form, the melting point B° C. of the barrier layer needs to be lower than A° C. (melting point of the first homogeneous material) to avoid re-melting of the first homogeneous material 51. In other words, the melting point of the barrier layer 50 needs to be lower than the first homogeneous material 51. The composition of the barrier layer is adjusted accordingly to have a melting point lower than the first homogeneous material. However, the melting point B° C. needs to be higher than room temperature or a normal storage temperature to avoid melting of the barrier layer at room temperature or a temperature of the normal storage conditions, which will result in losing the capability to act as a barrier and thus mixing with other layers.

In some embodiments, to avoid pressuring the surface of the first homogeneous material 51 from the adding process of the barrier layer due to direct spitting, spraying or discharging from the nozzle during the adding process, which will result in a rough surface of the first homogeneous material, the invention methods or devices comprise an invention nozzle that spits, sprays or discharges ingredients (e.g. a barrier layer) onto the inner wall 7 of a capsule, therefore reducing the pressure onto the surface of the first homogeneous material 51. Referring to FIGS. 5A and 5B, a barrier layer 50 is sprayed on a releasing point R of the wall 7, which has a distance d from the surface of the homogeneous material. In certain embodiments, R is about 2 mm to about 3 mm. In some embodiments, the spraying angle theta is about 0 to 60 degrees. In certain embodiments, the spraying angle theta is about 10 to 45 degrees. In certain embodiments, the spraying angle theta is about 10 to 40 degrees (FIG. 5B). The spraying angle theta is defined as the degree between the line L (from the nozzle spraying point S to the release point R of the inner wall) and the horizontal line from the nozzle spraying point S to the inner wall 7. One of the skilled in the art would readily recognize that the length and/or the size of the invention nozzle and the design of the tip or the releasing point of the invention nozzle can be varied in accordance with the specification of targeted invention capsules. The design shown in FIG. 5 is a non-limited example.

In some embodiments, if pressuring the surface of the first homogeneous material 51 from the adding process of the barrier layer due to direct spitting, spraying or discharging from the nozzle is not an issue, a conventional nozzle that direct spraying on the surface of the homogeneous material is used.

Step 865: cool down the barrier layer 50 to a temperature lower than B° C. via a cooling means. The purpose of this step is to ensure a solid form of the barrier layer 50 to avoid mixing with the next adding material (i.e. the second homogeneous material 52). When the temperature is lower than B° C., the barrier layer 50 becomes solid and has less chance to mix with the next adding. The cooling means is the same as one in step 855. The adding of the second component (i.e., the barrier layer 50) is completed after steps 860 and 865.

Step 850: add a second homogeneous material 52 to the body 2 of a capsule. In certain embodiments, the second homogeneous material 52 is added in a solid form such as solid block, micro capsules, granules, or powdery solid such as powder (condition 1). In certain embodiments, the second homogeneous material 52 is added in a liquid or semi-solid form (condition 2). In certain embodiments, the homogeneous material 52 in a liquid or semi-solid form during the adding process remains a form of liquid or semi-solid at room temperature or a normal storage temperature (condition 2a). In certain embodiments, the homogeneous material 52 in a liquid or semi-solid form during the adding process becomes solid block at room temperature or a normal storage temperature (condition 2b); in other words, the second homogeneous material 52 is in a semi-solid or liquid form at high temperature (e.g. higher than 35° C.).

For example, when adding the second homogeneous material 52 under condition 2b and the melting point of the second homogeneous material 52 is C° C., step 870 further includes a step to raise temperature above C° C. Furthermore, because the second homogeneous materials 52 is added on the surface of the cured (solidified) barrier layer 50, the melting point C° C. of the second homogeneous material 52 needs to be lower than B° C. (melting point of the barrier layer 50) to avoid re-melting of the barrier layer 50. In other words, the melting point of the second homogeneous material 52 needs to be lower than the barrier layer 50. As a result, based on steps 850, 860, and 870, A° C.>B° C.>C° C.; in other words, the material with the higher melting point needs to be added first. For example, a three-layer capsule comprising Zaleplon was prepared in accordance with the invention method. The three-layer capsule consists of two layers of homogeneous materials that comprise Zaleplon (i.e. the first homogeneous material and the second homogeneous material) and a layer of barrier that does not comprise Zaleplon. The barrier layer was made of paraffin wax. Both first and second homogeneous materials are solid block at room temperature. The temperature for adding the first homogeneous material was 75 to 80° C., where the material was fluid and easily added into a capsule. The barrier layer consisted 100% paraffin wax, which required a adding temperature at about 60 to 65° C. The adding temperature of the second homogeneous material was 55 to 60° C. In summary, to reach the melting points of the first homogeneous material 51, the barrier layer 50 and the second homogeneous layer 52, steps 850, 860 and 870 include further the heating steps to heat the homogeneous material, the barrier layer and the second homogeneous layer and the temperature range is between room temperature (e.g. 25° C.) and 80° C.

Another example of making a three-layer capsule is as follows. A vancomycin containing first homogeneous layer (comprising sodium alginate and polyethylene glycol glycerides) was first added into the body of capsules in a liquid form at raised temperature. The barrier layer (comprising paraffin wax) was then added on top of the first homogeneous material after it cooled down (from 70° C. to room temperature). The second homogeneous material comprising vancomycin and PEG1500 and polyethylene glycol glycerides was then added on top of the barrier layer after it cooled down to room temperature.

After step 870, step 875 may be selected to cool the second homogenous material 52. In some embodiments, the second homogeneous material is cooled down to room temperature or a temperature suitable for storage. The adding of the second homogeneous material 52 (i.e., the third component) is completed after steps 870 and 875. Step 880 includes mounting of the cover body 3 over the opening 4 of the body 2 to complete the exemplary invention process of manufacturing a multi-layer capsule. Step 890 includes discharging the multi-layer capsules and the capsules with incomplete fillers. The capsules with incomplete fillers are determined, for example, by their weights with weight measuring devices. When the weight of a capsule is not within the spec, the capsule will automatically be excluded; this process distinguishes the complete filled, incomplete filled and empty capsules where the later two types of capsules are excluded.

In some embodiments, the present invention provides a bio-friendly and safe barrier layer 50 to separate the first homogeneous material 51 and the second homogeneous material 52. In certain embodiments, the first homogeneous material 51 comprises one or more active pharmaceutical ingredients (APIs). In certain embodiments, the second homogeneous material 52 comprises one or more active pharmaceutical ingredients. In certain embodiments, the first and second homogeneous materials comprise one or more active pharmaceutical ingredients. The barrier layer 50 prevents characteristic changes of liquation (melting) between layers (i.e. the homogenous materials) and/or interactions of the APIs between the first homogeneous material and the second homogeneous material when both comprise APIs (same or different APIs). For example, without the barrier layer, the APIs may permeate between layers due to the concentration differences, thus changing the desired drug effects. In addition, any other effects cause by each other of the first homogeneous material 51 and second homogeneous material 52 can be avoided by a barrier layer 50.

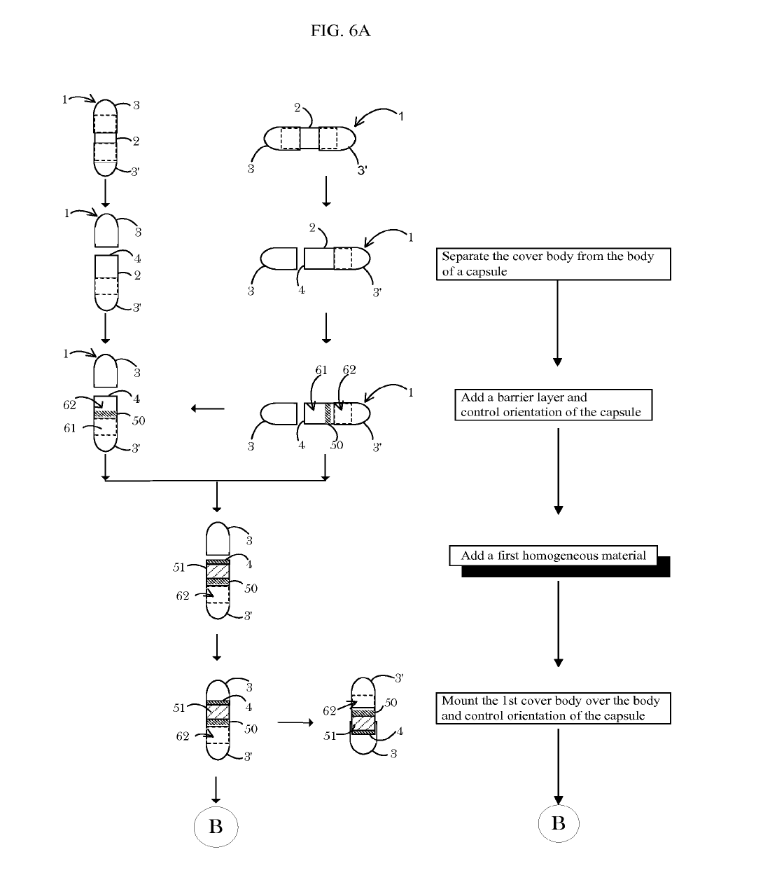

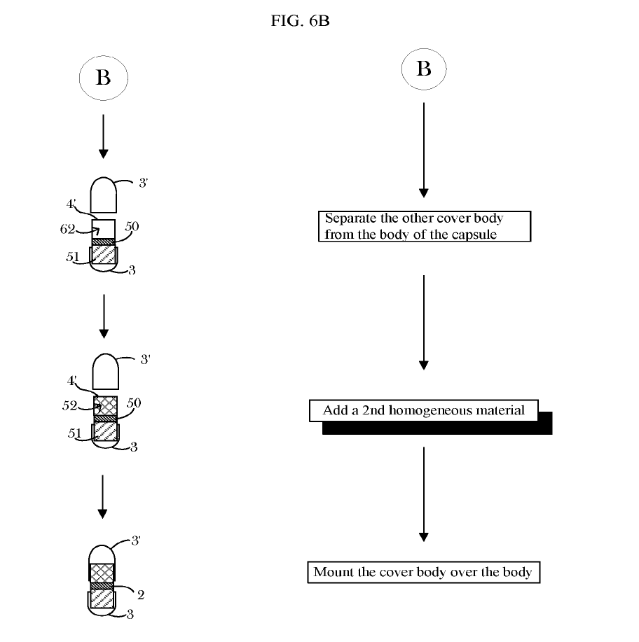

Other exemplary multi-layer capsules and methods of preparing same are shown in FIGS. 6A and 6B where a barrier layer 50 is added first and separates the internal space into two. Unlike the exemplary capsules and method of preparing same illustrated in FIG. 4, where the first and the second homogenous materials are introduced via the same opening 4, the body 2 herein has the openings 4 and 4′. As such, a cover body 3 which mounts over the opening 4 and a cover body 3′ which mounts over the opening 4′ are used. Thus, the first homogeneous material 51 is introduced via the opening 4 and the second homogeneous material 52 is introduced via the opening 4′. Consequently, two orientation adjusting steps are required.

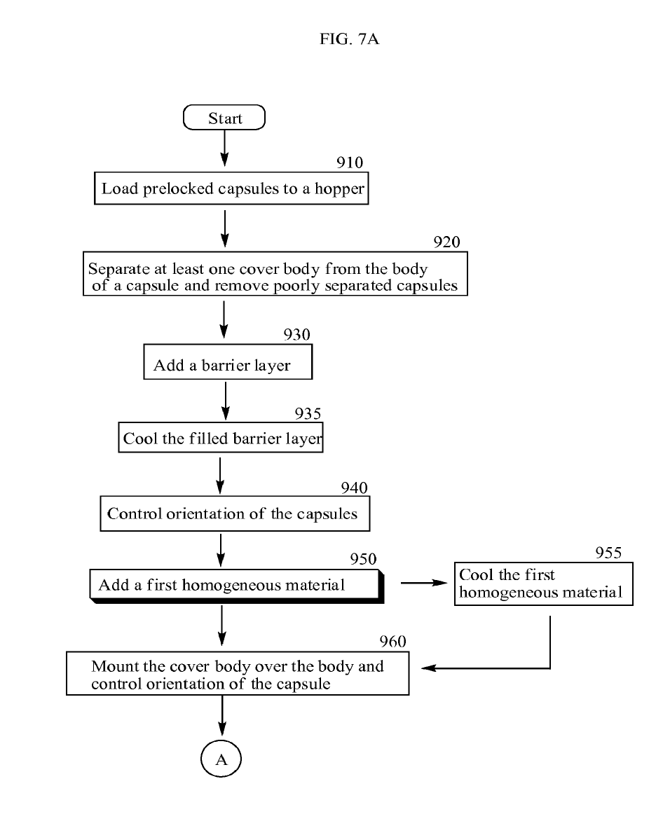

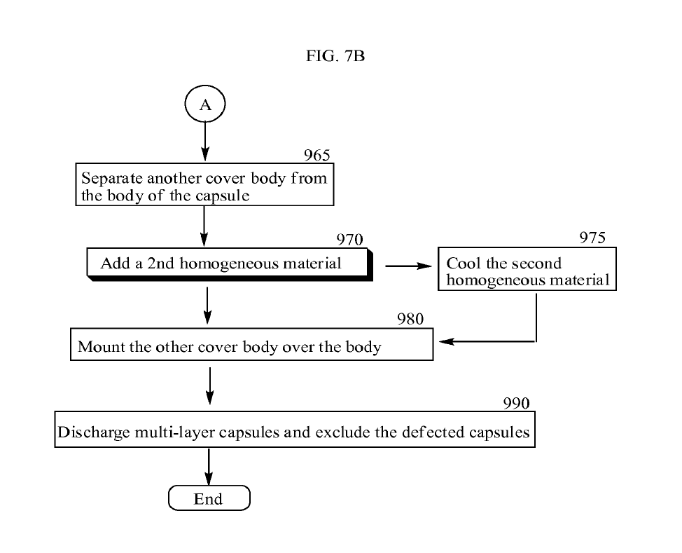

For example, referring to the procedure diagrams of FIGS. 7A-7B, step 910 includes putting prelocked capsules into the hopper. Step 920 includes separating at least one cover body from the body 2 and removes the capsules with poor separation. Step 930 includes adding of a barrier layer 50. The barrier layer may be formed directly in the internal space connecting to the inner wall or formed by adding into the internal space on top of a temporarily pre-installed stent (not shown in diagram 6A). The principal adding order is the same as stated before where the material with the higher melting point should be added first. Since in this example the barrier layer 50 is added first, the first and second homogeneous materials need to have lower melting points than the barrier layer if they are added in a semi-solid or liquid form. As such, the composition (and thus the melting point) of the barrier layer 50 is pre-determined in step 930 based on the melting points of the first and second homogeneous materials. The barrier layer needs to have a higher melting point than room temperature or a normal storage temperature so the barrier layer will not melt at room temperature or at a storage temperature that results in losing its function for layer separation and mixing with other layers.

Step 935: cool down the barrier layer 50. If a temporarily pre-installed stent is used in step 930, the stent is removed in step 935 before commencing next step. Step 940: control the orientation of the capsules. This is the first orientation adjustment to make axis of the body 2 perpendicular to the horizontal plane allowing the opening 4 of the body to be in a level and up position (also see FIG. 6A). Step 950 includes adding of the first homogeneous material 51; step 955 includes cooling of the first homogeneous material 51. Step 960 includes mounting the first cover body 3 over the opening 4 of the body 2 and rotate the orientation of the capsule 180 degree (continue FIG. 7A to 7B). Step 965 includes dismounting the second cover body 3′. Regarding step 960, the orientation adjustment is required to make the opening 4′ (opposite opening of the opening 4) to be in a level and up position after dismounting of the cover body 3′ in step 965. Step 970 includes adding of the second homogeneous material 52; step 975 includes cooling of the second homogenous materials 52. Step 980 includes mounting the second cap 3′ over the opening 4′ of the body 2; step 990 includes discharging the complete filled capsules and excluding the empty or incomplete filled capsules.

Thus, this example also provides an alternative adding order of the first and the second homogeneous materials. For example, when the first or second homogeneous material is micro-capsules, granules, powdery solid, semi-solid, or liquid during the adding steps and at room temperature or a temperature of normal storage conditions, the procedure can apply to preparation of the invention multi-layer capsules where, for example, a first homogeneous material 51 is added in a semi-solid or liquid form and a second homogeneous material 52 is micro-capsules, granules, or powder solid; or both the first and second homogeneous materials are added in a liquid form; or both materials are added in a semi-solid form. That is because the procedure does not require adding a barrier layer in a semi-solid or liquid form onto the surfaces of a micro-capsules, granules, powdery solid, semi-solid, or liquid layer (the implementation is not easy).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A multi-layer capsule comprising at least two layers of homogeneous materials and at least one barrier layer where each layer of homogenous material comprises the same or different biopharmaceutical ingredient, and wherein each layer of homogeneous material has the same or different form of liquation as the other layers.

2. The multi-layer capsule of claim 1, wherein said biopharmaceutical ingredient is an active pharmaceutical ingredient or a dietary supplemental ingredient.

3. A multi-layer capsule comprising:
a body comprising at least one opening and an internal space;
at least one cover body to mount or dismount over the at least one opening of the body;
a barrier layer disposed in the internal space of the body to separate the first compartment and the second compartment wherein the barrier layer is solid at room temperature and semi-solid or liquid at a temperature higher than 35° C.

4. The multi-layer capsule of claim 1 or claim 3, wherein the barrier layer comprises mineral oil and paraffin wax in a weight ratio between 0 to 4.

5. The multi-layer capsule of claim 3, wherein the thickness of the barrier layer accounts for about 5% to 25% of the body length of the capsule.

6. The multi-layer capsule of claim 3, wherein the body has two openings and two cover bodies to mount or dismount over each of the openings of the body.

7. The multi-layer capsule of claim 3, wherein the first compartment comprises a first homogeneous material and the second compartment comprises a second homogeneous material.

8. The multi-layer capsule of claim 7, wherein the first or second homogeneous material is in a solid form selected from solid block, micro capsules, granules, and powdery solid at room temperature.

9. The multi-layer capsule of claim 8, the first or second homogeneous material being in a solid block form wherein said solid block is characterized as a semi-solid or liquid at a temperature higher than 35° C.

10. The multi-layer capsule of claim 9, wherein the first or second homogeneous material comprises at least one hot melt excipient that is semi-solid or liquid at a temperature higher than 35° C.

11. The multi-layer capsule of claim 7, wherein the first or second homogeneous material is in a liquid form.

12. The multi-layer capsule of claim 11, wherein the liquid comprises a suspension.

13. The multi-layer capsule of claim 7, wherein the first or second homogeneous material is in a semi-solid form at room temperature.

14. The multi-layer capsule of claim 9, wherein the first homogeneous material has a melting point higher than the melting point of the barrier layer, and the barrier layer has a melting point higher than the melting point of the second homogenous material.

15. The multi-layer capsule of claim 9, wherein the barrier layer has a melting point higher than the melting point of the first homogeneous material and the second homogenous material.

16. A method of manufacturing a multi-layer capsule, the method comprising
providing a capsule comprising a body, and at least one cover body wherein the body has an internal space and at least one opening;
adding a first homogeneous material, a second homogeneous material, and a barrier layer to the internal space of the body wherein the barrier layer is added before or after the first homogeneous material; and
mounting the at least one cover body over the at least one opening of the body, wherein the first and second homogeneous material is separated by the barrier layer.

17. The method of claim 16, further comprising a capsule orientation controlling step that makes the axis of the body and the at least one cover body perpendicular to the horizontal plane, allowing at least one opening of the body to be in a level and up position.

18. The method of claim 16, further comprising dismounting the at least one cover body to expose at least one opening.

19. The method of claim 16, wherein the adding step further comprising heating the first homogeneous material, the second homogeneous material, and the barrier layer to be in a semi-solid or liquid form.

20. The method of claim 19, wherein the heating is between 25° C. to 85° C.

21. The method of claim 19, wherein the adding step further comprising cooling the first homogeneous material, the second homogeneous material, and the barrier layer to be in a solid or solid block form.

22. The method of claim 16, wherein the barrier layer comprises mineral oil and paraffin wax in a weight ratio between 0 to 4.

23. The method of claim 16, wherein the thickness of the barrier layer accounts for about 5% to 25% of the body length.

24. The method of claim 16, wherein the adding of a barrier layer after the first homogeneous material comprises spraying the barrier layer to a releasing point of the inner wall, wherein the distance between the releasing point and the top of the homogeneous material is about 2 mm to about 3 mm.

25. The method of claim 23, wherein the spraying of the barrier layer is in an angle about 10 degrees to about 40 degrees between a line defined from a nozzle spraying point to the releasing point of the inner wall and the horizontal line from the nozzle spraying point to the inner wall.

26. The method of claim 16, wherein the first, second or the barrier layer with higher melting point is added first.
